(19) United States
(12) Patent Application Publication
(10) Pub. No.: US 2017/0176448 A1
(43) Pub. Date: Jun. 22, 2017

(54) Health And Wellness Testing Kit For Home Use

(71) Applicant: Emilie DeAngelo, Somers Point, NJ (US)

(72) Inventor: Emilie DeAngelo, Somers Point, NJ (US)

(21) Appl. No.: 14/976,639

(22) Filed: Dec. 21, 2015

(51) Int. Cl. G01N 33/66; C12Q 1/54; G09B 23/28; G01N 33/84

(52) U.S. Cl. CPC G01N 33/66; G01N 33/84; C12Q 1/54; G09B 23/28

(57) ABSTRACT

The present invention relates to a health and wellness testing and lifestyle adjustment advice kit, and the like, and more particularly to a health and wellness testing kit for home use with accompanying instructions for testing results interpretation and lifestyle adjustment thereafter. Embodiments of the kit include pH testing materials and glucose testing materials for analyzing a user's urine sample, and informational and advice material (both written and on digital video) for reaching optimum urine pH values and glucose values.

### BRIEF DESCRIPTION OF THE INVENTION

The present invention relates to health and wellness testing and lifestyle adjustment advice, and the like, and more particularly to a health and wellness testing kit for home use with accompanying instructions for testing results interpretation and lifestyle adjustment thereafter.

### CROSS-REFERENCES TO RELATED APPLICATIONS

None.

### STATEMENTS AS TO THE RIGHTS TO INVENTIONS MADE UNDER FEDERALLY SPONSORED RESEARCH OR DEVELOPMENT

Not applicable.

### REFERENCE TO A “SEQUENCE LISTING,” A TABLE, OR A COMPUTER PROGRAM LISTING APPENDIX SUBMITTED ON A COMPACT DISK

Not applicable.

### BACKGROUND OF THE INVENTION

The present invention relates to health and wellness testing and lifestyle adjustment advice, and more particularly to a health and wellness testing kit for home use with accompanying instructions for testing results interpretation and lifestyle adjustment thereafter.

Existing technology allows for pH level testing and glucose level testing by individuals through over the counter (OTC) methods. The relative health and wellness of the human body can be ascertained through analysis of one's urine—this is referred to in the art as urinalysis. By testing the pH of a person's urine sample and by testing for the presence of glucose in a person's urine, a person can assess their own wellness and make lifestyle (and/or nutritional) adjustments depending on the results. But presently, persons interested in monitoring their wellness have to combine OTC testing supplies with their own wellness research, intuition, and charting methods in order to make such lifestyle and nutritional adjustments. There is a need in the art for a ready-to-use OTC home kit combining everything necessary for pH and glucose testing with information on the meaning of pH values and the meaning of the presence of glucose along with suggestions for lifestyle and nutritional adjustments.

pH testing can be accomplished through urinalysis using commercially available urine test strips. Commercial brands of urine test strips measure pH in incremental units between pH 5 and pH 9. Commonly, a double indicator system is used to display a visual change based on the pH reading of the urine sample. Double indicator systems utilize two chemical compounds, each compound affected at different pH levels. Methyl red produces a color change from red to yellow in the range of pH 4 to pH 6. Bromothymol blue changes from yellow to blue between pH 6 and pH 9. In practice, this double indicator system causes a urine test strip to display orange at pH 5, progressing through red to yellow to green and then dark blue as it approaches pH 9.

Glucose level testing by urinalysis is also known in the art. Urine test strips utilize a double sequential enzyme reaction to determine if glucose is present in the urine sample. The enzyme glucose oxidase catalyzes the formation of gluconic acid and hydrogen peroxide from the oxidation of glucose. A second enzyme, peroxidase, catalyzes the reaction of hydrogen peroxide with a potassium iodide chromogen to oxidize the chromogen. This second oxidation changes the color from green a lower present levels of glucose to brown at higher present levels of glucose.

It would be advantageous to combine pH and glucose testing with charting supplies for tracking over time and information on the meaning of pH values and the meaning of the presence of glucose along with suggestions for lifestyle and nutritional adjustments.

### BRIEF DESCRIPTION OF THE SEVERAL VIEWS OF THE DRAWING

FIG. 1 illustrates a general overview of the herein disclosed health and wellness testing kit for home use in accordance with the present invention;

FIG. 2a illustrates a top-down view of an exemplary embodiment of the carrying case of the herein disclosed health and wellness testing kit for home use in accordance with the present invention;

FIG. 2b illustrates a top-down view of an exemplary embodiment of the carrying case in use to hold several components of the herein disclosed health and wellness testing kit for home use in accordance with the present invention;

FIG. 3a illustrates a first side of an exemplary embodiment of a pH table of the herein disclosed health and wellness testing kit for home use in accordance with the present invention; and

FIG. 3b illustrates a second side of an exemplary embodiment of a pH table of the herein disclosed health and wellness testing kit for home use in accordance with the present invention.

### DETAILED DESCRIPTION OF THE INVENTION

The present invention is directed towards an apparatus that combines home testing of a person's bodily health and/or wellness with instructions for interpreting the test results and suggestions for lifestyle and nutritional adjustments intended to improve future results of the bodily tests. The apparatus will mostly be referred to as a kit throughout the specification and claims, however it is to be understood that any combination of the herein disclosed and described components, whether referred to as a “kit” or by alternative terminology such as “combination”, “supplies”, “materials”, “collection”, etc. are intended to be included within the description of the present invention. As such, the herein disclosed health and wellness testing kit for home use may alternatively be referred to as a health testing kit, a health test kit, a wellness testing kit, a wellness test kit, a home wellness kit, a home testing kit, or a kit of home testing for wellness and health.

While the present invention is mostly described as being for home use, it is to be understood that the presently described combination can be utilized by persons in any setting outside of a doctor's office or hospital. For example, the herein described health and wellness testing kit can be used at a person's place of work, such as an office, or at a public place, such as a public restroom or public park. As such, the present invention is not to be read as limited only to use in a person's home.

The herein disclosed health and wellness kit for home use may include one or more pH tests for determining a pH value of a urine sample, one or more glucose tests for determining a presence value of glucose in the urine sample, and an informational brochure. The informational brochure may include a meaning of the pH value, an optimum pH value, a meaning of the presence value of glucose, an optimum glucose value, one or more suggestions for obtaining the optimum pH value, and one or more suggestions for obtaining the optimum presence value of glucose. The health and wellness kit for home use may additionally include an informational recorded digital video in a fixed medium capable of being presented on a display screen. The informational digital video may include the optimum pH value, a second meaning of the pH value, a second set of one or more suggestions for obtaining the optimum pH value, the optimum presence value of glucose, a second meaning of the presence value of glucose, and a second set of one or more suggestions for obtaining the optimum presence value of glucose. The health and wellness kit for home use may additionally include a pH table for analyzing the pH value. The pH table may include a first side displaying a plurality of possible pH values and a plurality of possible meanings corresponding to the possible pH values and a second side displaying one or more suggestions for achieving a different pH testing value. The one or more suggestions for achieving different pH testing values may include a list of acidity foods for lowering a person's pH testing value and a list of alkaline foods for increasing a person's pH testing value. The health and wellness kit for home use may additionally include a progress chart (or charting table) for recording a plurality of pH values over time and a plurality of presence values of glucose over time. The health and wellness kit for home use may additionally include a set of urine sample cups. And the health and wellness kit for home use may include a carrying case, within which all the above described components can be conveniently stored and/or carried. The carrying case may be plastic, and may have a protruding handle and one or more clasps for snapping shut. The plastic carrying case may be designed so as to provide space for storing the one or more pH tests, the one or more glucose tests, the informational brochure, the informational recorded digital video, the pH table, the progress chart, and the set of urine sample cups.

Some embodiments of the present invention may further include pH drops for alkalizing water. These pH drops can be stored in a small vessel, and the health and wellness testing kit carrying case can be designed so as to additionally store the pH drops vessel. Other bodily testing apparatus and methods, such as tests for various blood tests known in the art can additionally be included in the herein disclosed health and wellness testing kit for home use. For example, tests for the presence and level of fatty acids (Omega 3 and Omega 6) in a person's blood can be included, and the carrying case can be designed so as to additionally store such testing apparatus.

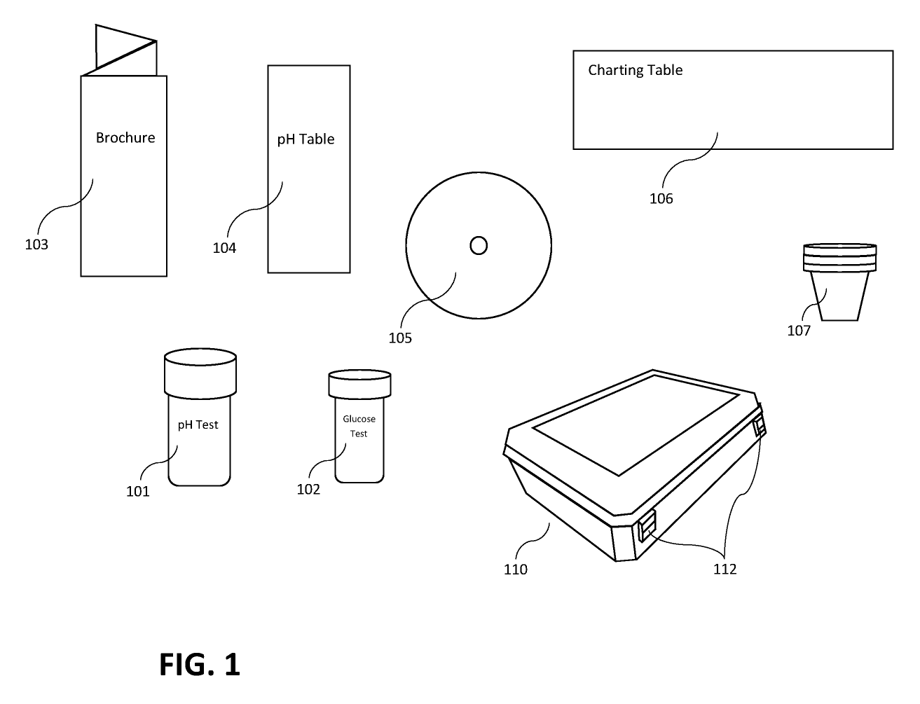

As illustrated in FIG. 1, the herein disclosed health and wellness testing kit for home use may include a pH test 101. pH test 101 may be urine test strips, which may be stored in a bottle. Such a urine test strip bottle may contain two or more of the urine test strips, may include child-safety features, and may be printed with instructions for utilizing the urine test strips to test urine samples. pH test 101 urine test strips may, for example, utilize a double indicator system to display visually apparent color changes based on a pH reading of a urine sample. The health and wellness testing kit for home use may also include glucose test 102. Glucose test 102 may also be urine test strips, and such, may also be stored in a bottle. Such a glucose urine test strip bottle may contain two or more urine test strips, may include child-safety features, and may be printed with instructions for utilizing the urine test strips to test urine samples. Glucose test 101 urine test strips may, for example, utilize a double sequential enzyme reaction to determine if glucose is present in the urine sample. Alternatively, glucose test 101 may be a blood sample test, or any other glucose test or testing method known in the art.

As also illustrated in FIG. 1, the herein disclosed health and wellness testing kit for home use may additionally include informational brochure 103. Informational brochure 103 may include written information presenting information to a user regarding the meaning of pH values obtained via pH test 101 and the meaning of glucose presence values obtained via glucose test 102. Informational brochure 103 may explain the general nature of the pH of urine samples, and may include one or more suggestions for lifestyle and nutritional adjustments that a user may make to obtain different pH value readings when utilizing pH test 101 or different glucose value readings when utilizing glucose test 102. The one or more suggestions for lifestyle and nutritional adjustments that a user may make to obtain different pH value readings when utilizing pH test 101 or different glucose value readings when utilizing glucose test 102 may be referred to as a pH advise set, a glucose advise set, or a pH and glucose advice set. Informational brochure 103 may, for example, be a tri-fold laminated paper brochure, as is known in the art. But informational brochure 103 may alternatively be any sort of written material capable of presenting a plurality of items of information to a user in an easily readable format. For example, informational brochure may be a booklet containing two or more pages, or may be written material printed on heavy card-stock (one-sided or two-sided).

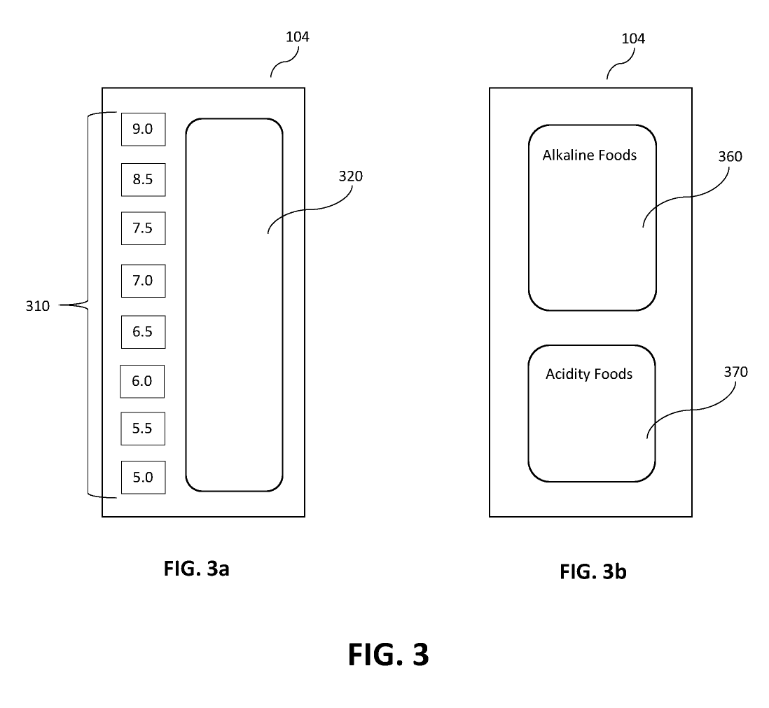

The herein disclosed health and wellness testing kit for home use may additionally include pH table 104. pH table 104 may include printed written material and may, for example, be laminated heavy card-stock. Alternatively, pH table 104 can be any sort of paper or plastic capable of presented written information to a user, as is known in the art. The contents of pH table 104 will be further described below in discussing FIG. 3.

The herein disclosed wellness testing kit may additionally include digital video 105, as illustrated in FIG. 1. Digital video 105 may be any sort of informational recorded digital video capable of being presented on a display screen for a user. For example, digital video 105 may be a DVD capable of playing on a standard DVD player either attached to a computer, a monitor, or a television, as is known in the art. Alternatively, digital video 105 may be a digital video file (such as an.gif, .fly, .wmv, .mpeg, or any other file type known in the art) stored on a thumb drive or a SD card, or any other digital media storage device known in the art. In an alternative embodiment, digital video 105 can be a website address linking to a page that displays such a digital video file at a user's request.

Digital video 105 may include identical, or substantially similar, information as is presented on informational brochure 103. Alternatively, digital video 105 may instead present additional information, such as more detailed information regarding the meaning of pH values, optimum pH values, the meaning of glucose presence values, optimum glucose presence values, and additional advice for a user to achieve, or come closer to, those optimum pH values and optimum glucose presence values.

The herein disclosed wellness testing kit may additionally include a charting table 106, as can be seen in FIG. 1. Charting table 106 may be utilized to chart one or more pH values over time, so that a user is able to track changes in pH value reading of their urine samples taken over time. For example, a user may take a urine sample every day and use pH test 101 to determine the pH value of that urine sample each day. The user can write his or her pH value each day on charting table 106, and thereby track changes in pH over some period of time. Charting table 106 may be printed with a grid, or a series of columns, to facilitate such periodic tracking. Testing can be performed daily, weekly, months, or at an alternative frequency desired by the user, and charting table 106 can be designed to accommodate any such frequency. Charting table 106 can be heavy stock paper, plastic, or any such appropriate material as is known in the art. Charting table 106 may additionally be capable or recording a plurality of glucose presence values obtained periodically over time by a user utilizing glucose test 102.

The health and wellness testing kit may additionally include urine sample cups 107. Urine sample cups 107 may be one or more cups of an appropriate size for collected a user's urine sample, as is known in the art. For example, urine sample cups 107 may be a stack of several 3 ounce dixie-type cups. In an alternative embodiment, urine sample cups 107 may be one reusable cup, intended to be washed between uses by a user.

The health and wellness testing kit may additionally include a carrying case 110. Carrying case 110 may be cardboard, plastic, metal, or any other material appropriately sturdy so as to be capable of storing and carrying several health and wellness testing items. For example, carrying case 110 may be designed so as to store and carry pH test 101, glucose test 102, informational brochure 103, pH table 104, digital video 105, charting table 106, and/or urine sample cups 107. In alternative embodiments, carrying case 110 can be designed so as to store and carry additional tests, informational materials, or associated wellness products. For example, carrying case 110 may additionally store and carry pH drops for water alkalizing.

Carrying case 110 may have a top lid and a bottom section for holding health and wellness items, and may include clasps 112. An alternative embodiment of the herein disclosed wellness testing kit may include a handle attached to carrying case 110. Such a handle may be approximately the size of one average human hand, and may protrude from a side of carrying case 110. Clasps 112 may be one or more clasps for snapping shut the lid of carrying case 110 to the bottom section. There are many possible sizes, shapes, and designs that are possible and appropriate for storing and carrying wellness items, and all such sizes, shapes, and designs are intended to be described and included herein.

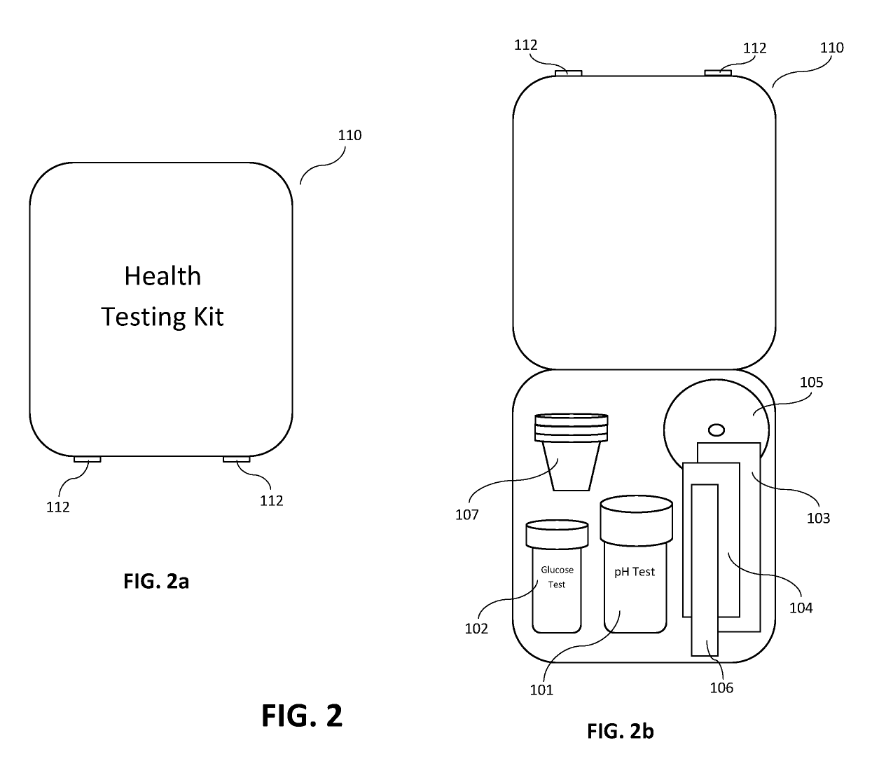

FIG. 2, which is comprised of FIG. 2a and FIG. 2b, illustrates carrying case 110 in both its closed state (FIG. 2a) and its open state with several wellness items enclosed (FIG. 2b). As is illustrated in FIG. 2b, health and wellness testing items such as pH test 101, glucose test 102, informational brochure 103, pH table 104, digital video 105, charting table 106, and/or urine sample cups 107 can be stored and carried inside of carrying case 110. The position of each health and wellness testing item inside carrying case 110 is shown only as an example, and is not intended to be limiting. As discussed above, additional wellness items, such as pH drops for water alkalizing or Omega 3 fatty acid testing apparatus for example, may also be stored and carried in carrying case 110.

As illustrated in FIG. 3, pH table 104 may include a first side (shown in FIG. 3a) and a second side (shown in FIG. 3b). The first side of pH table 104 may include a plurality of pH values 310. For example, pH values 310 may display pH values between 5.0 and 9.0, such as 5.0, 5.5, 6.0, 6.5, 7.0, 7.5, 8.5, and 9.0 (8.0 may be omitted because it may be an uncommon pH value and therefore may be less useful). Other configurations of pH values 310 are possible, known in the art, and all are intended to be included herein. pH value information 320 may be located close in proximity to pH values 310. pH value information 320 may include written information and/or advice corresponding to each of the pH values displayed in pH values 310. Alternatively, pH value information 320 may only include two or more items of written information and/or advice corresponding to some lesser fraction of pH values 310. For example, pH value information may only include three separate items of information or advice, one corresponding to pH values 5.0 through 6.5, one corresponding to pH values 6.5 through 7.5, and one corresponding to pH values 7.5 through 9.0. An example of pH value information 320 may be: “If your reading is showing too much acid then take a look at the other side and begin incorporating more alkaline foods into your life” (corresponding to pH values 5.0 through 6.0), “Most water is usually 7 . . . pH drops will drive up alkaline for you to 8-9” (corresponding to pH value of 6.5), “A reading between 6.5 & 7.0 may not appear to be a big deal, however it can be” (corresponding to pH values between 6.5 and 7.0) “7.2-7.3 is ideal” (corresponding to pH values between 7.0 and 7.5), “raw is best —cooked makes more acidity” (corresponding to pH value of 7.5), and “Over doing it . . . get your numbers down! See reverse side for some less alkaline foods. Too much alkaline is not health either. Must remember balance is key!” (corresponding to pH values of 8.5 and 9.0).

A second side of pH table 104 is illustrated in FIG. 3b. The second side of pH table 104 may include information and/or advice for adjusting a user's lifestyle and/or nutrition in order to alter pH values obtained using pH test 101. For example, the second side of pH table 104 may include a list of Alkaline Foods 360, and a list of Acidity Foods 370. Alkaline Foods 360 may be a written list of food items, it may be one or more pictures or drawings of food items, or it may be a combination of written items and pictured/drawn items. Similarly, Acidity Foods 370 may be a written list of food items, it may be one or more pictures or drawings of food items, or it may be a combination of written items and pictured/drawn items. In an alternative embodiment, the second side of pH table 104 can include additional written (or pictured) advice for achieving adjusted pH values, or a combination of food listings and advice.

pH table 104 has been described as a single sheet, preferable on heavy card stock or other sturdy material, but pH table 104 may alternatively be a small booklet, a tri-fold brochure, or any other informational display method or apparatus as is known in the art.

While the present invention has been illustrated and described herein in terms of a preferred embodiment and several alternatives, it is to be understood that the techniques described herein can have a multitude of additional uses and applications. Accordingly, the invention should not be limited to just the particular description and various drawing figures contained in this specification that merely illustrate a preferred embodiment and application of the principles of the invention.

What is claimed is:

1. A wellness testing kit comprising:
one or more pH tests for determining a pH value of a urine sample;
one or more glucose tests for determining a presence value of glucose in the urine sample;
an informational brochure, the informational brochure including a meaning of the pH value and a meaning of the presence value of glucose; and
a carrying case, the carrying case designed to hold the one or more pH tests, the one or more glucose tests, and the informational brochure.

2. The wellness testing kit as recited in claim 1, wherein the one or more pH tests are urine test strips.

3. The wellness testing kit as recited in claim 1, wherein the one or more glucose tests are urine test strips.

4. The wellness testing kit as recited in claim 1, further comprising:
a pH drops vessel, the pH drops vessel containing a plurality of pH water alkalizing drops.

5. The wellness testing kit as recited in claim 1, wherein the carrying case is formed from hard plastic.

6. The wellness testing kit as recited in claim 1, wherein the carrying case includes a protruding handle approximately as large as an average adult human hand.

7. The wellness testing kit as recited in claim 1, wherein the carrying case includes one or more clasps for snapping shut.

8. The wellness testing kit as recited in claim 1, wherein the written information includes an optimum pH value and a pH advice set, the pH advice set including one or more advice items for achieving the optimum pH value.

9. The wellness testing kit as recited in claim 1, wherein the written information includes an optimum presence value of glucose and a glucose advice set, the glucose advice set including one or more advice items for achieving the optimum glucose value.

10. The wellness testing kit as recited in claim 1, further comprising:
a set of urine sample cups.

11. The wellness testing kit as recited in claim 10, wherein the set of urine sample cups includes one or more disposable cups.

12. The wellness testing kit as recited in claim 1, further comprising:
an informational recorded digital video capable of being presented on a display screen and including a second meaning of the pH value and a second meaning of the presence value of glucose.

13. The wellness testing kit as recited in claim 12, wherein the informational recorded video is stored on a computer readable DVD.

14. The wellness testing kit as recited in claim 12, wherein the second meaning of the pH value includes an optimum pH value and a pH advice set, the pH advice set including one or more advice items for achieving the optimum pH value, and wherein the second meaning of the presence value of glucose includes an optimum presence value of glucose and a glucose advice set, the glucose advice set including one or more advice items for achieving the optimum glucose value.

15. The wellness testing kit as recited in claim 1, further comprising:
a pH table for analyzing the pH value.

16. The wellness testing kit as recited in claim 15, wherein the pH table includes a first side displaying a plurality of possible pH values and a plurality of possible meanings corresponding to each of the possible pH values and a second side displaying one or more pH lowering suggestions and one or more pH increasing suggestions.

17. The wellness testing kit as recited in claim 16, wherein the one or more pH lowering suggestions include an acidity foods list, and wherein the one or more pH increasing suggestions include an alkaline foods list.

18. The wellness testing kit as recited in claim 15, further comprising:
a progress chart for recording a plurality of pH values over time and a plurality of presence values of glucose over time.

19. A wellness testing kit comprising:
one or more pH tests for determining a pH value of a urine sample;
one or more glucose tests for determining a presence value of glucose in the urine sample;
an informational brochure, the informational brochure including a meaning of the pH value, an optimum pH value, a meaning of the presence value of glucose, an optimum glucose value, one or more suggestions for obtaining the optimum pH value, and one or more suggestions for obtaining the optimum presence value of glucose;
an informational recorded digital video in a fixed medium capable of being presented on a display screen and including the optimum pH value, a second meaning of the pH value, a second set of one or more suggestions for obtaining the optimum pH value, the optimum presence value of glucose, a second meaning of the presence value of glucose, and a second set of one or more suggestions for obtaining the optimum presence value of glucose;
a pH table for analyzing the pH value, the pH table including a first side displaying a plurality of possible pH values and a plurality of possible meanings corresponding to the possible pH values and a second side displaying one or more pH lowering suggestions including a list of acidic foods and one or more pH increasing suggestions including a list of alkaline foods;
a progress chart for recording a plurality of pH values over time and a plurality of presence values of glucose over time;
a set of urine sample cups; and
a plastic carrying case with one or more clasps for snapping shut, the plastic carrying case designed to hold the one or more pH tests, the one or more glucose tests, the informational brochure, the informational recorded digital video, the pH table, the progress chart, and the set of urine sample cups.

20. The wellness testing kit as recited in claim 19, further comprising:
a pH drops vessel, the pH drops vessel containing a plurality of pH water alkalizing drops; and wherein the plastic carrying case is also designed to hold the pH drops vessel.
